(19) United States
(12) Patent Application Publication
(10) Pub. No.: US 2021/0052348 A1
(43) Pub. Date: Feb. 25, 2021

(54) An Augmented Reality Surgical Guidance System

(71) Applicant: Medivation AG, Brugg (CH)

(72) Inventors: Tobias Schwägli, Solothurn (CH); Jan Stifter, Schneisingen AG (CH)

(73) Assignee: Medivation AG, Brugg (CH)

(21) Appl. No.: 16/963,826

(22) PCT Filed: Jan. 16, 2019
(86) PCT No.: PCT/EP2019/050997

§ 371 Date: Jul. 21, 2020

(30) Foreign Application Priority Data
Jan. 22, 2018 (CH) 00064/18

(51) Int. Cl. A61B 90/00; A61B 90/92; A61B 34/20

(52) U.S. Cl. CPC A61B 90/36; A61B 90/92; A61B 2017/00734; A61B 90/39; A61B 2034/2048; A61B 34/20

(57) ABSTRACT

An augmented reality surgical guidance system includes an augmented reality device and a plurality of mobile surgical tracking devices, including at least a first mobile surgical tracking device and a second mobile surgical tracking device, wherein at least one of the first or second mobile surgical tracking devices is connected to an object. The first mobile surgical tracking device includes a marker, a sensor and a control unit. The sensor of the first mobile surgical tracking device is configured to track the position of the second mobile surgical tracking device or the augmented reality device. The sensor is connected to the control unit to provide positional information data of the second mobile surgical tracking device to the control unit. The control unit includes a transmission unit configured to transmit the positional information data to the augmented reality device and the augmented reality device includes an imaging device and a display.

The invention is related to an augmented reality surgical guidance system including a plurality of mobile surgical tracking devices and an augmented reality device. The mobile surgical tracking device can be attached to the patient and/or any surgical instruments to provide accurate tracking of the relevant surgical parameters. This tracking information is transferred to the augmented reality device. The augmented reality device can overlay the surgical scene with instrument locations, 3D anatomical models of the patient, medical images of the patient based on the mobile surgical tracking device position, in particular within the field of view of the augmented reality device.

Current augmented reality surgical intervention systems use an external optical tracking system that can track the surgical tools position, the patient position and virtual reality display position which requires all elements to be equipped with optical markers like reflective spheres. Such a setup requires always a line of sight to all the markers and the augmented reality display which is often difficult in a surgical setup. The position of the fiducial marker has to be registered to the augmented reality displays position. Alternatively, the tracking system of the augmented reality display is used to accurately track the surgical instruments and patient's position. This solution has the drawback that the augmented reality tracking system may not be accurate enough to provide the critical accuracy needed for computer assisted surgical interventions for example in orthopedics, spine surgery or other surgical fields. A customized augmented reality system would be required to embed a high accuracy tracking system into the augmented reality device as the currently available augmented reality systems are consumer electronic devices with a limited accuracy.

Mobile, instrument or patient mountable tracking systems are described for surgical navigation for example in US2008319491 A1 and US 20130274633 A1. The tracking system of US2008319491 A1 is part of a surgical navigation system and locates and tracks arrays in real-time. The positions of the arrays are detected by cameras and displayed on a computer display. The tracking system is used to determine the three-dimensional location of the instruments which carry markers serving as tracking indicia. The markers may emit light, in particular infrared light or reflect such light. The light is emitted or reflected to reach a position sensor for determining the position of the instrument. The specific anatomical structure of the patient can be characterized by a limited number of landmarks, which can be used to generate a virtual patient specific instrument. The patient specific instrument can include a tracking device, e.g. a reference array. The position of the reference array is thus known and can be used to position the patient specific instrument virtually on the display. Due to the fact, that rigid reference arrays can be obtained, the patient's bone structure can be tracked without the need of additional rigid array markers. The navigation system automatically recognizes the position of the reference array relative to the patient's anatomy. A system for performing a computer-assisted hip replacement surgery is disclosed in document US2013/0274633. The system comprises a pelvis sensor, a broach sensor and a femur sensor coupled to the respective bone or broach structure. The position of the sensors is recorded during the surgery by a processing device. The processing device can perform a femoral registration by measuring an orientation between the broach sensor and the femur sensor. The processing device can display a fixed target frame and a track frame, which can be matched by adjusting the positions of the bone and broach structures and when the matching position is reached, the change in leg length and a change in offset can be calculated. Each of the sensors can be configured as an optical reader or a beacon. Another mobile surgical tracking system is described in U.S. Pat. No. 8,657,809 B2. This tracking system is non-invasively attached to the patient's head for a ENT surgery. In this setup, a single camera is used to track marker elements mounted on an instrument to track the instruments position relative to the patient's head. A mobile surgical tracking system according to EP3162316A1 is mounted to patient's anatomy with the help of a patient specific mating surface to allow a defined mounting position of the tracking system, requiring no registration of the tracking systems position to the patient anatomy. According to CH00005/17 the mobile surgical tracking system or parts of it are equipped with fiducial marker elements that can be detected in medical imaging pre- and/or intra-operatively.

For tracking the surgical instrument position in relation to the patient and the augmented reality display a tracking system must be used. In WO 2017066373 A1 the basic configuration of such an augmented reality display system to overlay a virtual model of the patient with the real patient is disclosed either by using an external tracking system by using a sensor mounted in the surgical room or a sensor mounted on the augmented reality display system.

In US 20140022283 A1 a configuration using an external tracking system is disclosed that also tracks the position of a semitransparent plate that is used as an augmented reality display. The tools and display are equipped with optical markers in order to be tracked by a stereo-camera system placed in the surgical room. A projector is used to visualize the information on the display. This setup also requires an external tracking system and the mounting of plate that serves as the augmented reality display may be difficult in the sterile surgical environment.

In document US 20060176242 an augmented reality device presents an augmented image to the user of a surgical scene. The tracking of the surgical scene is either made by an external stereo-vision camera system or by a tracking system attached to the augmented reality display device. The position of the display in relation to the surgical scene and surgical instruments is tracked by the external tracking system or display mounted tracking system.

The documents WO 2010067267, U.S. Pat. No. 7,774,044 B2 describe head mounted surgical augmented reality systems that incorporate an optical tracking system. An optical tracking system suited to track optical markers on instruments and attached to the patient is incorporated into the head mounted surgical augmented reality system. The surgical augmented reality system can be used as a complete navigation system. However, the user must always have his view directed towards the patient to keep the markers to be tracked in sight. In some situations, it would be beneficial if tracking information would be available even if the user is not looking at the surgical site. Also, in some situations, the user may decide to use a conventional display to continue the surgery and the headset may be to heavy and uncomfortable to carry throughout the full procedure. Adding an accurate tracking system to track surgical instruments may result in a heavy and expensive head mounted augmented reality system.

The tracking systems built into augmented reality system are therefore not suited to provide accurate and reliable information about the surgical instrument positions within their field of view.

Therefore, there is a need in an improved augmented reality surgical guidance system. An augmented reality surgical guidance system is subject of claim 1. Further advantageous embodiments of the system are subject of the dependent claims.

If the term «for instance» is used in the following description, the term relates to embodiments or examples, which is not to construed as a more preferred application of the teaching of the invention. The terms “preferably” or “preferred” are to be understood such that they relate to an example from a number of embodiments and/or examples which is not to construed as a more preferred application of the teaching of the invention. Accordingly, the terms “for example”, “preferably” or “preferred” may relate to a plurality of embodiments and/or examples.

The subsequent detailed description contains different embodiments of the mobile surgical tracking system according to the invention. The mobile surgical tracking system can be manufactured in different sizes making use of different materials, such that the reference to a specific size or a specific material is to be considered as merely exemplary. In the description, the terms «contain», «comprise», «are configured as» in relation to any technical feature are thus to be understood that they contain the respective feature but are not limited to embodiments containing only this respective feature.

An augmented reality surgical guidance system comprising an augmented reality device and a plurality of mobile surgical tracking devices includes at least a first mobile surgical tracking device and a second mobile surgical tracking device. At least one of the first or second mobile surgical tracking devices is connected to an object. The first mobile surgical tracking device includes a marker, a sensor and a control unit. The sensor of the first mobile surgical tracking device is configured to track the position of the second mobile surgical tracking device or the augmented reality device. The sensor is connected to the control unit to provide positional information data of the second mobile surgical tracking device or the augmented reality device to the control unit. The control unit includes a transmission unit configured to transmit the positional information data to the augmented reality device. The augmented reality device or at least one of first or second mobile surgical tracking devices includes an imaging device and a display. The imaging device is configured to process an image of the object. The display is configured to overlay the image of the object with output information of at least one of the first or second mobile surgical tracking devices based on the positional information data in the image of the object.

As the mobile surgical tracking device is attached directly at the site of intervention and the instrument is in close range of the tracking system, there are no line of sight issues as with existing external optical tracking solutions. An advantage of the system is that when a mobile surgical tracking system is used in combination with the augmented reality system the implementation can be made simpler and lightweight and therefore also easier to wear during a full surgery.

In addition, tracking information is always available as the mobile surgical tracking system is directly attached to the patient and instruments with less line of sight issues. As no dedicated and accurate mobile surgical tracking system has to be built into the augmented reality device, therefore a consumer electronic device could be used in combination with the mobile surgical tracking system.

There are multiple advantages in combining a mobile surgical tracking system with an augmented reality device compared to existing implementations. The combination allows accurate tracking of relevant surgical parameters by the means of a mobile surgical tracking system directly attached to the patient's anatomy and surgical instruments with almost no line of sight issues. By using the tracking system in the augmented reality system an augmented reality based surgical guidance system can be implemented that can overlay navigation information and image data onto the patient's anatomy or relative the surgical tools positions.

The mobile surgical tracking device according to any of the embodiments is preferably lightweight to be mountable to a patient or fixed to an anatomical structure like a bone. Also, a small size is required not to interfere with imaging or other surgical tools.

According to an embodiment, the second mobile surgical tracking device can include a control unit, a sensor and a marker. The sensor of the second mobile surgical tracking device can be configured to track the position of the marker of the first mobile surgical tracking device or the augmented reality device. The sensor can be connected to the control unit to provide positional information data of the first mobile surgical tracking device to the control unit. The control unit can include a transmission unit configured to transmit the positional information data to the augmented reality device or to the first mobile surgical tracking device.

According to an embodiment, the plurality of mobile surgical tracking devices can be attached to a plurality of anatomical structures. Each mobile surgical tracking device can be configured as to be equipped only with a marker, thus with a trackable element so that each mobile surgical tracking device can act as a trackable device and each mobile surgical tracking device position can be determined by the augmented reality display device even if the mobile surgical tracking device doesn't contain a sensor or a control unit.

According to an embodiment, the augmented reality device includes a marker, a sensor and a control unit, such that any of the first or optionally any additional, e.g. the second or third, mobile surgical tracking device can track the augmented reality device.

The object can be one of a surgical instrument, a patient specific instrument or a patient's anatomical structure or a virtual 2D or 3D model of the patient's anatomical structure, a surgical room, a person, a patient's surface, an instrument geometry. According to an embodiment, the positional information data include coordinate 6D position data.

According to an embodiment, at least one of the mobile surgical tracking devices is equipped with an identification element, such as a special housing geometry and/or a housing coloring. The identification element can be detectable by a tracking system of the augmented reality device. The identification element can be used for distinguishing between different mobile surgical tracking devices, for instance the housings can include different colors or can include different geometrical elements or tags. The identification element can be a coding placed on the housings for improving tracking or identification.

According to an embodiment, the marker includes an optical marker element or a LED in a known configuration. In particular, the optical marker element includes one element of the group of lines, circles, mobile tags trackable by the augmented reality device. The optical marker element can be measured by the augmented reality device and be used to overlay information based on the measure positions.

The optical marker element can be detectable by the augmented reality device tracking system. The optical marker elements can be attached the mobile surgical tracking device at a known position.

According to an embodiment, the optical marker element is configured as a single or multiple faced tag including preferably one or more geometric elements. According to a further embodiment, the optical marker element can be the same or partially the same used by an optical measurement system of the mobile surgical tracking device. The optical marker elements can include one of specific coloring, optical surface properties or reflective material. The geometric element may include one of a line, circle, ellipse or a pattern detectable by using a computer vision algorithm.

According to a further embodiment the optical markers can be single or multiple LED's that are placed at a known position on the mobile tracking systems elements. Using single LED's, the augmented reality system can detect 2D position of mobile tracking system elements and show information based on this single LED positions which may be enough for certain applications. To more accurately track the full 6D position of the mobile surgical tracking system elements multiple LED's can be used in a known geometric configuration. This allows the augmented reality system to determine the 6DOF position of the elements and show augmented reality information at correct 3D location in relation to the patient's anatomy. One or multiple of the described LED's may be used by the mobile surgical tracking system and the augmented reality system for positional tracking. In an embodiment the two tracking systems are synchronized so that the LED's can be used by both systems for tracking.

The mobile surgical tracking device can contain fiducial marker elements for the direct registration of medical images to the coordinate frame of the tracking system and in combination with the augmented reality display device allow an overlay of these medical images with the actual patient's position.

The system can be used in the field of orthopedics, spine, cranial/neuro, ENT (ear, nose, throat), dental navigation or any other image guided surgical intervention. The mobile surgical tracking device can be used for image guided interventions where a CT or cone beam CT scan is acquired pre-operatively. The mobile surgical tracking device can be attached in a known positional relationship with respect to the patient close to the surgical field. According to this configuration, the scan can be made by integrating the integrated fiducial marker into the imaging volume. Thereby a direct registration of the imaging device coordinate frame to the patient coordinate frame is possible. Either the mobile surgical tracking device can be left on the patient until the surgical procedure is carried out or the mobile surgical tracking device can be fixed at the same location for the surgical intervention.

According to an embodiment, one of the first or second mobile surgical tracking devices or the augmented reality device includes a shadow imaging tracking. In particular, the shadow imaging tracking includes an optical grating or a mask above an imaging sensor to track the position of the marker. The mobile surgical tracking device can thus comprise an integrated optical tracking system. The optical tracking system can be implemented as a stereo- or multi-camera optical system. The optical tracking system can be used for tracking an active or a passive marker. Such systems are known and well described but based on the required optics and computation tasks for tracking, an integration to a very small form factor is not straightforward. Alternatively, a single camera tracking system can be provided, as this system would require less space, but the achievable accuracy of this system is limited.

The integrated optical tracking system can comprise a shadow imaging tracking, e.g. using a shadow mask above an imaging sensor in order to track the position of a marker equipped with three or more LEDs in a known configuration. In a preferred embodiment a shadow imaging technology is used as tracking system in the mobile surgical tracking device. This tracking system only requires an optical sensor, for example a CCD chip with a shadow mask on top of it and the computation can be implemented by a small size embedded system. It is possible to integrate all components in a single chip for further reduction of the possible form factor. The trackable elements require at least 3 LEDs in a known spatial configuration that are measured by the shadow imaging system. With the single LED position, the tracking system can compute the 6D position of the trackable element. Another advantage of the shadow imaging tracking is its large opening angle of 120° or more, which is a substantial advantage for close range measurements. The principle of shadow imaging is described in EP 2793042 A1 and its integration with surgical instruments is described EP15192564 A1, which are incorporated by reference in their entirety into this application.

According to an embodiment, the mobile surgical tracking device comprises multiple integrated optical tracking systems to allow measurement in multiple directions, whereby each of the integrated optical tracking systems can comprise a measurement volume, whereby at least one of the optical tracking systems can be separate or at least two of the optical tracking systems can be overlapping.

According to an embodiment, one of the mobile surgical tracking devices or the augmented reality device includes an accelerometer or an inertial measurement unit to generate tracking data. These tracking data can be used together with optical tracking information to determine the position of the mobile surgical tracking devices. In particular, a combination of a positional tracking, e.g. based on a single or multiple LED, together with data obtained from the inertial measurement unit or accelerometer can be used to determine the position of mobile surgical tracking devices more accurately. In particular, the tracking data of the accelerometer can be used if high frame-rate tracking is required for example to adjust a displayed image based on a changed head pose as the optical tracking frame-rate may not be sufficient for this purpose.

According to an embodiment, the display is mono- or stereoscopic and can be configured to display the positional information as 2D or 3D overlay.

A semitransparent display can be positioned between the user and the operative field or a mobile device like a tablet or a mobile phone can overlay the live camera image with the output information. In particular, the display comprises a movable display. The display may be one of a computer including a display or a smartphone or a tablet device.

According to an embodiment, the augmented reality device comprises a head mounted display. A head mounted display or augmented reality helmet/glasses is worn by the user and the information is displayed directly in front of the user's eye on a semitransparent display. The head mounted display can be a mono- or stereoscopic display. The mobile surgical tracking device can be used to track the position of surgical instrument and display their position and the patient's anatomy overlaid to the real surgical site on the display the augmented reality device. When worn by the user, it is beneficial if the augmented reality device is battery driven and can work autonomously. The augmented reality device can be completely integrated into glasses or a helmet worn by the user, e.g. the surgeon. It is also possible that a control unit containing a battery is worn by the user for example with a belt to reduce the weight of a head mounted part of the augmented reality device so as to keep the head mounted part of the augmented reality device as light as possible for the user to wear.

According to an embodiment, the augmented reality device is configured to match the image of the object with the object. The augmented reality device can include a tracking sensor designed to track its position in relation to the surgical scene in real time and overlay the scene with relevant information. In particular, a high frame rate tracking using multiple sensors like stereo-vision, depth-camera and inertial sensors are provided. According to an embodiment, the imaging device includes a camera, whereby the camera can be a video-camera configured to provide a video.

The augmented reality device can comprise a control unit to calculate the position of the mobile surgical tracking device in the image. The display of the augmented reality device can display the position of the mobile surgical tracking device or the model of the anatomical structure generated from the images from an imaging device such as a camera, which can be combined with the patient's anatomical structure and/or the other mobile surgical tracking devices. The images or any anatomical structure model can be matched directly with the patient, in particular, the anatomical structure of the body part which has to be treated by the surgery. The information can be shown to the user through wearable smart glasses.

According to an embodiment, one of the first or second mobile surgical tracking devices can be attachable to a patient by means of a patient specific instrument attachable to a surface of a patient's anatomical structure.

The augmented reality device can include a coordinate system. The object can include a coordinate system. The first or second mobile surgical tracking devices include a first and second coordinate system. Any further or additional mobile surgical tracking devices can include further or additional coordinate systems.

The position of the coordinate systems of the first or second mobile surgical tracking devices and the coordinate system of the object in the coordinate system of the augmented reality device can be determined by the control unit of the augmented reality device based on the positional information data received from any of the first or second mobile surgical tracking devices and the object.

The position of the coordinate system of the augmented reality device in one of the coordinate systems of the respective mobile surgical tracking devices can determined by the respective control unit of the respective first or second mobile surgical tracking device if the positional information data from the augmented reality device is processed in the control unit of the respective mobile surgical tracking device.

According to an embodiment, the transmission unit comprises a wireless transmission unit. The wireless transmission unit can be configured as a wireless link. The tracking data can be transferred to the augmented reality display device that guides the surgical intervention over a wireless link as for example Bluetooth LE.

According to an embodiment, the at least one of the mobile surgical tracking devices and the augmented reality device is battery driven. The battery operation should allow for tracking during a surgery normally for at least some minutes up to several hours. The battery can be replaceable or rechargeable. A single use mobile surgical tracking device can be provided to be used for only one single surgery. For other applications, a resterilizable mobile surgical tracking device can be preferable. The highly integrated design of the mobile surgical tracking system according to any of the embodiments allows to produce a mobile surgical tracking device configured as a single use device.

According to an embodiment, the output information comprises an image or a text, including preferably one of a step in surgical workflow, an instruction how to assemble and use a surgical tool, a critical anatomical structure, a preoperative plan.

According to an embodiment, the mobile surgical tracking device is configured to track the position of the augmented reality device.

According to an embodiment, the transmission unit of one of the first or second mobile surgical tracking device is configured to transmit the augmented reality device position data to the augmented reality device. The tracking of the position of the augmented reality device relative to the mobile surgical tracking device can be implemented by the mobile surgical tracking device. The augmented reality device is in this case equipped with an optical marker that can be detected by the mobile surgical tracking device. The tracking data is then transferred to the augmented reality device. The augmented reality device can use this positional information to generate the augmented reality overlay based on the positional data. The augmented reality device can use the positional data of the mobile surgical tracking system as described above or in combination with its own tracking data. Sensor fusion algorithms can be applied to improve augmented reality tracking.

The advantage of an augmented reality surgical guidance system including a plurality of mobile surgical tracking devices in combination with the augmented reality device is that the mobile surgical tracking device can provide very accurate tracking of surgical instruments for measurements if high precision and reliability is required as the sensor and markers are directly attached to the patient or the instruments. The mobile surgical tracking device can be operated in very close range. To overlay information accurately with the patient and instrument's positions the augmented reality system can furthermore track one or more of the mobile tracking system elements and display surgical guidance information overlaid with their respective positions.

A head mounted display can provide different types of augmented reality implementations. This can be a simple 2D augmented reality where tracking data and navigation data as for example drill depth is displayed directly in the field of view of the user. A more advanced implementation features full stereoscopic augmented reality where information can be shown as virtual 3D object placed in the surgical scene relative to the patient. In such an implementation an overlay of medical images with patient anatomy providing a virtual look into the body are possible. It is possible to overlay the surgical instruments directly with navigational information or highlight critical instrument positions or anatomical structures.

In another embodiment the augmented reality device is configured as a mobile device such as a tablet. The augmented overlay is generated based on a video acquired by the camera of the augmented reality device or a camera attached to the augmented reality device. The augmented reality device can be used for a navigated intervention as a conventional display, requiring the additional functionality of an augmented reality device only for specific steps of the surgical procedure. The mobile device can either use the camera image to detect the location of the mobile surgical tracking device in the scene or can use additional tracking information, such as inertial or accelerometer measurements. In a further configuration the mobile device may be equipped with additional tracking hardware for example a stereo-camera or a depth sensing camera to enable the augmented reality display and tracking of the mobile surgical tracking device. In one embodiment the mobile device comprises a camera including an integrated optical measurement system such as shadow imaging system as described above or has such a system attached to it.

Besides the navigation information that can be calculated based on the surgical instruments position determined by the mobile surgical tracking system the augmented reality device can provide additional information to the user that can help in performing the surgical procedure safe and efficiently. Tracked objects can include elements of the surgical room, other persons, patients surface, instrument geometry. Additional information can be provided by the augmented reality device. Such information can also be shown for objects or parts of the patient anatomy not connected to the mobile surgical tracking system. Such information may include also patient information, vital signs and other relevant information for the surgical procedure. Based on the direction of view of the user, different information may be displayed. For example, when the user looks at any selection of instruments, such as a selection of instruments placed on an instrument table, instrument information for the selection of instruments can be displayed including information regarding single or multiple instruments of the instrument selection.

Based on the scene, in addition to the information presented to the user by the augmented reality display, such as like video feed or tracked spatial objects, additional information can be overlaid. The additional information can include one of a current step in surgical workflow, an instruction how to assemble and use a surgical tool, a critical anatomical structure or the preoperative plan. The augmented reality device can provide different views modes for the user to see different information.

The invention will be explained in more detail in the following with reference to the drawings. There are shown in a schematic representation in:

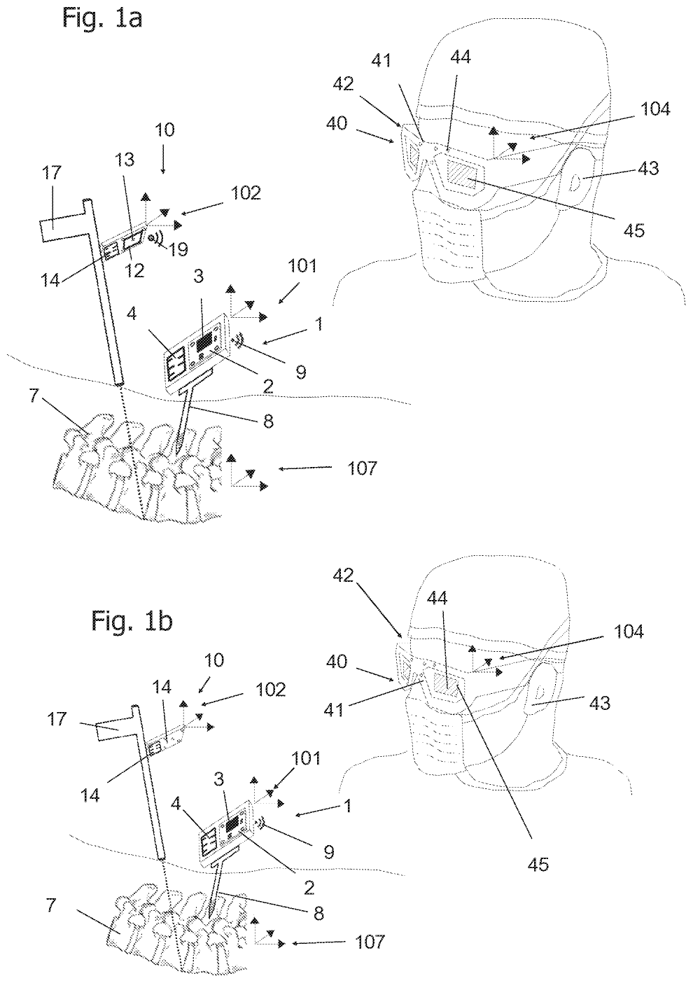

FIG. 1a a schematic view of an augmented reality surgical guidance system according to a first embodiment of the invention,

FIG. 1b a schematic view of an augmented reality surgical guidance system according to a second embodiment of the invention,

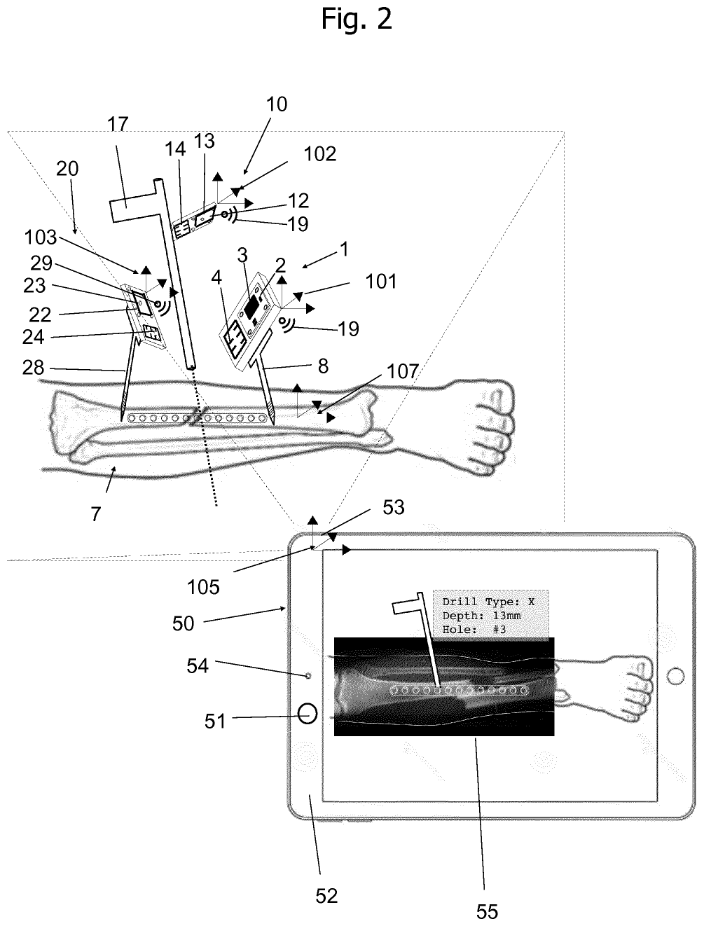

FIG. 2 a schematic view of an augmented reality surgical guidance system according to a third embodiment of the invention.

FIG. 1a shows a schematic view of an augmented reality surgical guidance system according to a first embodiment of the invention. The augmented reality surgical guidance system according to FIG. 1a comprises a first and second mobile surgical tracking device 1, 10 attached to the patient anatomy and an augmented reality device 40. The second mobile tracking device 10 is configured as a surgical instrument. The first mobile surgical tracking device 1 comprises a sensor 3, a marker 4, a control unit 2, including a computation unit, and a transmission unit 9. The second mobile surgical tracking device 10 comprises a sensor 13, a control unit 12, a marker 14 and a transmission unit 19. The augmented reality device 40 according to FIG. 1a is configured as a head mounted augmented reality device. A spine application is shown in FIG. 1a. The sensor 1 of the first mobile surgical tracking device 1 can track the 6D position of the marker 14 of the second mobile surgical tracking device 10. The marker comprises one of an optically detectable marker or an active LED. At least one of the first or second mobile surgical tracking devices 1, 10 or the augmented reality device 40 can include an imaging device 41, such as a single camera or stereo-camera setup that can track active or passive optical markers in space, such as the markers 4, 14.

At least one of the first or second mobile surgical tracking devices can include an optical tracking system, such as a shadow imaging system which can measure LED positions by a shadow projected using an optical grating in front of an optical sensor. By using a marker 4, 14 with multiple LED's a 6D position of the marker position can be computed by the respective control unit 2, 12.

The first or second mobile surgical tracking device 1, 10 can include a transmission unit 9, 19 can transmit data by a wireless link to the control unit 2, 12 and or directly to the augmented reality device 40. The mobile surgical tracking device 1, 10 can be either single use or resterilizable depending on the surgical application. Any one of the marker 4, 14, the sensor 3, 13, the control unit 2, 12, may be single use or may be resterilizable or vice versa.

At least one of the first or second mobile surgical tracking devices 1, 10 can be connected to an object 7, 17. The object 7 is a patient's anatomy to which the first surgical tracking device 1 is attachable. The object 17 is a surgical instrument, to which the second surgical tracking device 10 is attachable or attached. The first surgical tracking device 1 is fixed to the patient anatomy 7, here a bone structure of a patient's spine, using a fixation 8, in particular a pin fixation. Other fixations 8 to the patient are possible for example through a clamp, a base plate attached with screws or other known surgical fixation devices. It is also possible that one of the first or second mobile surgical tracking devices 1, 10 is fixed non-invasively to the patient's skin for example with adhesive tape.

The second mobile surgical tracking device 10 of FIG. 1a is configured as a surgical instrument, in particular a surgical tool, e.g. a drill guide to accurately drill holes for screw fixations. Other surgical tools like drills, saws, cut slots etc. can be tracked in a similar way.

The augmented reality device 40 includes a coordinate system 104. The object 7 includes a coordinate system 107, The first and second mobile surgical tracking devices 1, 10 include a first and second coordinate system 101, 102. The coordinate system 107 of the object 7, e.g. the anatomical structure of the patient, can be registered to the mobile surgical tracking device coordinate system 101 by a variety of known registration methods, for example a pointer-based registration method. The position of the coordinate systems 101, 102 of the first or second mobile surgical tracking devices 1, 10 and the coordinate system 107 of the object 7 in the coordinate system 104 of the augmented reality device 40 is determined by the control unit 42 of the augmented reality device 40 based on the positional information data received from any of the first or second mobile surgical tracking devices 1, 10 and the object 7. The position of the coordinate system 104 of the augmented reality device 40 in one of the coordinate systems 101, 102 of the respective mobile surgical tracking devices 1, 10 is determined by the respective control unit 2, 12 of the respective first or second mobile surgical tracking device 1, 10 if the positional information data from the augmented reality device 40 is processed in the control unit 2, 12 of the respective mobile surgical tracking device 1, 10.

The fixation 8 can include a patient specific attachment mating the anatomical surfaces to fix the mobile surgical tracking device 1 in a known position to object 7, thus the anatomical structure. The registration method can include an image-based registration method to register the patient anatomy using intra-operative imaging.

The first mobile surgical tracking device 1 can track the position of the second mobile surgical tracking device 10 relative to the object 7, which can be represented by pre- or intra-operatively acquired images or segmented anatomical 3D models. Instead of showing this information on a stationary computer screen, the augmented reality display device 40 can be used to display output information directly in the field of view of the user. The augmented reality device 40 or at least one of first or second mobile surgical tracking devices 1, 10 includes an imaging device 41 and a display 45. The imaging device 41 is configured to process an image of the object 7, 17. The display 45 is configured to overlay the image of the object 7, 17 with output information of at least one of the first or second mobile surgical tracking devices 1, 10 based on the positional information data in the image of the object 7, 17.

The output information of at least one of the first or second mobile surgical tracking devices 1, 10 can include the tracking information of the second mobile surgical tracking device 10 and the patient position, which is transmitted to the augmented reality device 40 by one of the first or second the mobile surgical tracking devices 1, 10. Pre- or intraoperatively acquired images and or segmented bone structures of the patient anatomy can be transferred from the imaging devices to the augmented reality display device or a computation unit that is part of this device. The control unit 42 of the augmented reality device 40 can determine in real time the positions of the first and second mobile surgical tracking devices 1, 10 with their respective coordinate systems 101, 102 in relation to the augmented reality device coordinate system 104. Using this information, the augmented reality device 40 can now show surgical guidance information directly in the field of view of the user using a semi-transparent display element 43.

The display 45 can be mono- or stereo-ocular showing information to only one eye or both. The type of information shown to the user can vary depending on the surgical application and accuracy of the tracking system. In one embodiment, basic information like calculated values can be shown next to a mobile surgical tracking device 1, 10, such as a surgical tool. For example, the actual drill depth could be displayed right beside the drill sleeve of a drilling tool. If a critical drilling depth is reached a warning could be shown directly at the tip of the sleeve indicating a critical value. If the augmented reality device 40 is able to accurately track the positions of the mobile surgical tracking devices 1, 10 in its coordinate system 104, the display 45 provide more sophisticated augmented reality functions by with overlaying the scene with medical images (e.g. X-Rays, CT, MR) or datasets of the patient allowing virtual view of structures inside the object 7. The tracked mobile surgical tracking devices 1, 10 can be embedded in the display 45 of the augmented reality device 40.

The first or second mobile surgical tracking device 1, 10 is equipped with markers 4, 14, in particular with additional optical markers or the same marker 4, 14 can be used by both the first or second mobile surgical tracking device 1, 10 and the augmented reality device 40. The first or second mobile surgical tracking device 1, 10 can be equipped with LED's. The position of the LED's can be tracked by the augmented reality device 40. In another configuration it is possible that the position of the augmented reality device 40 is tracked by one of the first or second mobile surgical tracking device 1, 10 and the augmented reality device 40 is equipped with a marker 44, e.g. a single LED or multiple LED's in a known configuration. The tracking information of the position of the augmented reality device 40 can be integrated into the coordinate system 104 of the augmented reality device 40. The respective mobile surgical tracking device coordinate system 101, 102 can be transmitted by a wireless link to the display 45 of the augmented reality device 40. The augmented reality device 40 can include a sensor 43, like for example a depth sensing camera, visible light stereo-camera system, accelerometer and or inertial measurement units. The sensor 43 can generate tracking sensor information as an output. The augmented reality device 40 can include a control unit 42 that is configured to process the positional information data and to generate the augmented reality overlay. The augmented reality device can include a marker 44. The first or second mobile surgical tracking device 1, 10 can track the augmented reality device 40. The positional information data can include tracking sensor information, which can be processed by the augmented reality device 40 by the control unit 42 using sensor fusion techniques to overlay output information, e.g. augmented reality information, as accurate as possible to the view of the user. The sensor 3, 13 of one of the first and second mobile surgical tracking devices 1, 10 can be equipped with an additional sensor as for example an accelerometer or an inertial measurement unit. The output information of the sensor or sensors 3, 13 can be submitted to the augmented reality device 40 to determine the position of the first and second mobile surgical tracking devices 1, 10 more accurately. Output information in particular additional output data may be displayed on the display 45 of the augmented reality device 40, whereby this output information is in particular present as an item in the field of view of the user. The output information can include one of a patient information, a critical vital signs information, an information about a surgical intervention, an information about a surgical technique. The display 45 could also provide output information to guide the user by displaying information about the next surgical step to execute or display the type of instrument and instructions how to assemble and use the instrument for the intended surgical step. Depending on the direction of the view of the user, different types of output information can be displayed on the display 45.

FIG. 1b shows a schematic view of an augmented reality surgical guidance system according to a second embodiment of the invention. This embodiment differs from the embodiment according to FIG. 1a in that no sensor nor a control unit is provided for the second mobile surgical tracking device 10. The second mobile surgical tracking device 10 is thus configured as a tracked device. The second mobile surgical tracking device includes a marker 14. FIG. 1b shows two different types of markers 14, which may be present alternatively or additionally, such as an optical marker or a LED. The second mobile surgical tracking device 10 can be tracked by the first mobile surgical tracking device 1 or the augmented reality device 40. However, the second mobile surgical tracking device 10 is not configured to track either the first mobile surgical tracking device 1, any further mobile surgical tracking device not shown in FIG. 1b nor the augmented reality device 40.

FIG. 2 shows a schematic view of an augmented reality surgical guidance system according to a third embodiment of the invention. The augmented reality surgical guidance system of FIG. 2 includes a first, second and a third mobile surgical tracking device 1, 10, 20 attached to the patient anatomy and an augmented reality device 50.

According to FIG. 2, the second mobile surgical tracking device 10 is configured as a surgical instrument. Any of the first or third mobile surgical tracking devices 1, 20 can also be configured as surgical instruments, which is not shown in the drawings. The first mobile surgical tracking device 1 comprises a sensor 3, a marker 4, a control unit 2, including a computation unit, and a transmission unit 9. The second mobile surgical tracking device 10 comprises a sensor 13, a control unit 12, a marker 14 and a transmission unit 19. The third mobile surgical tracking device 20 comprises a sensor 23, a control unit 22, a marker 24 and a transmission unit 29. The augmented reality device 50 according to FIG. 2 is configured as a mobile device, such as a tablet. The sensor 3 of the first mobile surgical tracking device 1 can track the 6D position of the marker 14 of the second mobile surgical tracking device 10 or the 6D position of the marker 24 of the third mobile surgical tracking device 20.

The augmented reality device 50 includes a mobile device including a video based augmented reality device on a tablet device.

The mobile surgical tracking devices 1, 10, 20 can be attached an object 7, such as multiple body parts of the patient, here two tibia bone fragments of a fractured bone. The object 7 is equipped with the first, second and third mobile surgical tracking systems 1, 10, 20. Fixations 8, 28 are provided for the first and third mobile surgical tracking systems 1, 20. The first mobile surgical tracking device 1 is attached to a first bone fragment. The second mobile surgical tracking device 10 is configured as a surgical instrument, e.g. a drill sleeve is equipped with LED's in a known arrangement to be tracked by the sensor 3, 23 of one of the first or third mobile surgical tracking devices or by the sensor 53 of the augmented reality device 50. On other body parts, additional mobile surgical tracking devices including markers or sensors can be attached. The third mobile surgical tracking device 20 comprises a marker 24 which includes a plurality of LED's. The third mobile surgical tracking device 20 is attached to a second bone fragment using a fixation 28. Each one of the mobile surgical tracking devices 1, 10, 20 can track the location any one of the other mobile surgical tracking devices. If the respective mobile surgical tracking device 1, 10, 20 is attached to the object 7, the location of the bone structures as well as the surgical instrument(s) in relation to each other can be determined. The transmission unit 9, 19, 29 is configured to transmit tracking data wirelessly to the augmented reality device 50. The augmented reality device 50 or at least one of first or second or third mobile surgical tracking devices 1, 10, 20 include an imaging device 51 and a display 55. The imaging device 51 is configured to process an image of the object 7, 17, wherein the display 45 is configured to overlay the image of the object 7, 17 with output information of at least one of first, second or third mobile surgical tracking devices 1, 10, 20 based on the positional information data in the image of the object 7, 17.

The augmented reality device 50 can include a control unit 52 that is configured to process the positional information data and to generate the augmented reality overlay. The positions of the one or more mobile surgical tracking devices can be tracked by the augmented reality device 50 to generate the augmented reality overlay on a live video captured by the imaging device 51, e.g. the rear camera of the augmented reality device 50. The markers 4, 14, 24 can be used to track the position of the respective mobile surgical tracking device 1, 10, 20. In addition, further markers, such as LED's can be used for tracking. The markers can include other geometric features of the mobile surgical tracking device suitable for obtaining the position thereof in the scene, e.g. the operating room. Information from different sources can be combined and shown on the display 55 to the user as an augmented reality image. The quality of the augmented reality image depends on the accuracy the measured positions of the mobile surgical tracking devices 1, 10, 20 and their respective coordinate systems 101, 102, 103 in the coordinate system 105 of the augmented reality device 50. The overlaid information can just contain some critical information like the current drill depth of a drill bit close to the instrument in use. In this case, only a rough position of the mobile surgical tracking devices may be required. If the augmented reality device 50 is configured to track the mobile surgical tracking device 1, 10, 20 with higher accuracy and full 6DOF position, a more advanced augmented reality image can be displayed on the display 55 by overlaying for example pre- or intra-operatively acquired images like radiography with the surgical site. This allows the user to virtually look into the patient's body and critical structures/tissue may be highlighted or shown using the augmented reality device 50. Also, it is possible to show an image of a standard surgical navigation system on the display 55, if the augmented reality image is only needed for certain critical surgical procedural steps and not throughout the full procedure. The mobile surgical tracking devices attached to the object 7, 17, e.g. the patient or instrument, may also be used to provide positional information for surgical navigation.

The position of the coordinate systems 101, 102, 102 of the first, second or third mobile surgical tracking devices 1, 10, 20 and the coordinate system 107 of the object 7 in the coordinate system 105 of the augmented reality device 50 is determined by the control unit 52 of the augmented reality device 50 based on the positional information data received from any of the first, second or third mobile surgical tracking devices 1, 10, 20 and the object 7. The position of the coordinate system 105 of the augmented reality device 50 in one of the coordinate systems 101, 102, 103 of the respective mobile surgical tracking devices 1, 10, 20 is determined by the respective control unit 2, 12, 22 of the respective first, second or third mobile surgical tracking device 1, 10, 20 if the positional information data from the augmented reality device 50 is processed in the control unit 2, 12, 22 of the respective mobile surgical tracking device 1, 10, 20. It is not required that all of the mobile surgical tracking devices are disposed with a respective control unit. Any of the first, second or third mobile surgical tracking devices can be substituted with a mobile surgical tracking device without control unit, such as the second mobile surgical tracking device disclosed in FIG. 1b.

The augmented reality surgical guidance system according to any of preceding embodiments thus combines a plurality of mobile surgical tracking devices with an augmented reality device. The mobile surgical tracking devices can be attached to the patient or to surgical instruments. The augmented reality surgical guidance system provides accurate tracking of the relevant surgical parameters, this tracking information is transferred to the augmented reality device. The augmented reality device can overlay the surgical scene with instrument locations, 3D anatomical models of the patient, medical images of the patient based on the mobile tracking systems position within the field of view of the augmented reality device. It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or the claims refer to at least one of an element or compound selected from the group consisting of A, B, C . . . and N, the text should be interpreted as requiring only one element from the group, not A plus N, or B plus N, etc.

1. An augmented reality surgical guidance system including an augmented reality device and a plurality of mobile surgical tracking devices, including at least a first mobile surgical tracking device and a second mobile surgical tracking device,
wherein the first or second mobile surgical tracking devices are connected to an object,
wherein the first mobile surgical tracking device includes a marker, a sensor and a control unit,
wherein the sensor of the first mobile surgical tracking device is configured to track a position of the second mobile surgical tracking device or the augmented reality device,
wherein the sensor is connected to the control unit to provide positional information data of the second mobile surgical tracking device or the augmented reality device to the control unit,
wherein the control unit includes a transmission unit configured to transmit the positional information data to the augmented reality device and
wherein the augmented reality device or at least one of the first or second mobile surgical tracking devices includes an imaging device and a display,
wherein the imaging device is configured to process an image of the object, and
wherein the display is configured to overlay the image of the object with output information of at least one of first or second mobile surgical tracking devices based on the positional information data in the image of the object.

2. The system according to claim 1, wherein the second mobile surgical tracking device includes a second control unit, a second sensor and a second marker, wherein the second sensor of the second mobile surgical tracking device can be configured to track a further position of the marker of the first mobile surgical tracking device or the augmented reality device, wherein the second sensor is connected to the second control unit to provide additional positional information data of the first mobile surgical tracking device to the second control unit, wherein the second control unit includes a second transmission unit configured to transmit the additional positional information data to the augmented reality device or to the first mobile surgical tracking device.

3-5. (canceled)

6. The system according to claim 1, wherein the augmented reality device includes a third marker, a third sensor and a third control unit such that the first or optionally any additional mobile surgical tracking device can track the augmented reality device, wherein the augmented reality device includes a coordinate system, wherein the object includes an object coordinate system, wherein the first or second mobile surgical tracking devices include a first and second coordinate system, wherein the position of the first and second coordinate systems of the first or second mobile surgical tracking devices and the object coordinate system of the object in the coordinate system of the augmented reality device is determined by the third control unit of the augmented reality device based on the positional information data or the additional positional information data received from any of the first or second mobile surgical tracking devices and the object.

7. The system according to claim 1, wherein the object is one of a surgical instrument, a patient specific instrument or a patient's anatomical structure or a virtual 2D or 3D model of the patient's anatomical structure, a surgical room, a person, a patient's surface, an instrument geometry.

8. The system according to claim 1, wherein the positional information data include coordinate 6D position data.

9. The system according to claim 1, wherein at least one of the mobile surgical tracking devices is equipped with at least one of a special housing geometry or a housing coloring.

10. The system according to claim 1, wherein the first or second marker includes an optical marker element or a LED in a known configuration, wherein the optical marker element includes one element of a group of lines, circles, mobile tags trackable by the augmented reality device.

11. (canceled)

12. The system according to claim 1, wherein one of the first or second mobile surgical tracking devices or the augmented reality device includes a shadow imaging tracking.

13. The system according to claim 12, wherein the shadow imaging tracking includes an optical grating or a mask above an imaging sensor to track the position of the marker.

14. The system according to claim 1, wherein one of the first or second mobile surgical tracking devices or the augmented reality device includes an accelerometer or an inertial measurement unit to generate tracking data.

15. The system according to claim 1, wherein the display is mono- or stereoscopic and can be configured to display the positional information as 2D or 3D overlay or comprises a movable display or wherein the augmented reality device comprises a head mounted display.

16-17. (canceled)

18. The system according to claim 1, wherein the augmented reality device is configured to match the image of the object with the object.

19. The system according to claim 1, wherein the imaging device includes a camera, in particular a video-camera configured to provide a video.

20. The system according to claim 1, wherein one of the first or second mobile surgical tracking devices is attachable to a patient by means of a patient specific instrument attachable to a surface of a patient's anatomical structure.

21-22. (canceled)

23. The system according to claim 6, wherein the position of the coordinate system of the augmented reality device in one of the first and second coordinate systems of the respective first or second mobile surgical tracking device is determined by the control unit or the second control unit of the respective first or second mobile surgical tracking device if the positional information data or the additional positional information data from the augmented reality device is processed in the control unit or the second control unit of the respective first or second mobile surgical tracking device.

24. The system according to claim 1, whereby the transmission unit comprises a wireless transmission unit.

25. The system according to claim 1, wherein at least one of the first or second mobile surgical tracking devices or the augmented reality device is battery driven.

26. The system according to claim 1, wherein the output information comprises an output image or a text, including one of a step in surgical workflow, an instruction how to assemble and use a surgical tool, a critical anatomical structure, or a preoperative plan.

27. The system according to claim 1, wherein the first or second mobile surgical tracking device is configured to track the position of the augmented reality device.

28. The system according to claim 1, wherein the transmission unit or the second transmission unit of one of the first or second mobile surgical tracking devices is configured to transmit the augmented reality device position data to the augmented reality device.
